(19) United States
(12) Patent Application Publication
(10) Pub. No.: US 2024/0262287 A1
(43) Pub. Date: Aug. 8, 2024

(54) SYSTEMS AND METHODS FOR DETECTING POTENTIAL VEHICLE OPERATOR INCAPACITATION BASED ON OPERATOR PHYSIOLOGICAL STATE DATA

(71) Applicant: HONEYWELL INTERNATIONAL INC., Charlotte, NC (US)

(72) Inventors: Thea Feyereisen, Minneapolis, MN (US); Gang He, Morris Plains, NJ (US); Hicham Atassi, Brno (CZ); Jan Ciganek, Brno (CZ); Bohdan Blaha, Brno (CZ); Nasiruddin Mohammed, Plymouth, MN (US)

(73) Assignee: HONEYWELL INTERNATIONAL INC., Charlotte, NC (US)

(21) Appl. No.: 18/165,694

(22) Filed: Feb. 7, 2023

(51) Int. Cl. B60Q 9/00; A61B 5/00; A61B 5/024; A61B 5/11; B63B 79/15; B64D 45/00

(52) U.S. Cl. CPC B60Q 9/00; A61B 5/02438; A61B 5/1118; A61B 5/6804; A61B 5/681; A61B 5/721; B63B 79/15; B64D 45/00; A61B 2503/22; A61B 2560/0252

(57) ABSTRACT

Systems and methods are provided for detecting potential vehicle operator incapacitation based on operator physiological state data. Raw operator physiological state data is received from a biosensing wearable device of an operator of a vehicle. The raw operator physiological state data includes raw operator actigraphy state data. Contextual motion data is received from at least one environmental motion sensor. The contextual motion data is associated with movement of the vehicle. A motion filter mask is generated based on the contextual motion data. The motion filter mask is applied to the raw operator actigraphy state data to filter the contextual motion data from the raw operator actigraphy state data to generate actual operator actigraphy state data. A determination is made regarding whether the actual operator actigraphy state data is less than an actigraphy threshold. An operator incapacity alert is issued based on the determination.

### TECHNICAL FIELD

The present invention generally relates to vehicle operations and more particularly relates to systems and methods for detecting potential vehicle operator incapacitation based on operator physiological state data.

### BACKGROUND

There is an increased interest in the use of the physiological states of vehicle operators to improve the overall safety of transportation systems. The physiological states of a vehicle operator may provide insight into whether the vehicle operator may be potentially incapacitated. In many instances, the vehicle operator wears non-invasive biosensing wearable devices that are configured to provide operator physiological state data that can be used to estimate the physiological state of an operator. An example of operator physiological state data is actigraphy. Actigraphy is a measurable variable associated with a body part of the vehicle operator that can be obtained from an accelerometer of the biosensing wearable device. Actigraphy is an indicator of the intensity of movement of the body part being monitored by the wearable device and is considered to be a reliable indicator of potential vehicle operator incapacitation.

In many cases, when actigraphy is used to assess the physiological state of the vehicle operator, environmental dynamics associated with the movement of the vehicle may negatively impact the reliability of the operator physiological state data received from the biosensing wearable device of the vehicle operator. For example, a large bump on a road or turbulence encountered during an aircraft flight may be misinterpreted as vehicle operator motion, when the source of that motion is just an artifact of an environmental state of vehicle motion. Environment dynamics may impact the accuracy of operator actigraphy measurements and lead to unreliable detection of potential vehicle operator incapacitation.

Hence there is a need for systems and methods for removing the influence of environmental dynamics on sensed operator physiological data prior to the use of the operator physiological data in the detection of potential operator incapacitation.

### BRIEF SUMMARY

This summary is provided to describe select concepts in a simplified form that are further described in the Detailed Description. This summary is not intended to identify key or essential features of the claimed subject matter, nor is it intended to be used as an aid in determining the scope of the claimed subject matter.

An exemplary embodiment of a system includes a processor and a memory. The memory includes instructions that upon execution by the processor, cause the processor to: receive raw operator physiological state data from a biosensing wearable device of an operator of a vehicle, the raw operator physiological state data including raw operator actigraphy state data; receive contextual motion data from at least one environmental motion sensor, the contextual motion data being associated with movement of the vehicle; generate a motion filter mask based on the contextual motion data; apply the motion filter mask to the raw operator actigraphy state data to filter the contextual motion data from the raw operator actigraphy state data to generate actual operator actigraphy state data; determine whether the actual operator actigraphy state data is less than an actigraphy threshold; and issue an operator incapacity alert based on the determination.

An exemplary embodiment of a method includes: receiving raw operator physiological state data from a biosensing wearable device of an operator of a vehicle, the raw operator physiological state data comprising raw operator actigraphy state data; receiving contextual motion data from at least one environmental motion sensor, the contextual motion data being associated with movement of the vehicle; generating a motion filter mask based on the contextual motion data; applying the motion filter mask to the raw operator actigraphy state data to filter the contextual motion data from the raw operator actigraphy state data to generate actual operator actigraphy state data; determining whether the actual operator actigraphy state data is less than an actigraphy threshold; and issuing an operator incapacity alert based on the determination.

Furthermore, other desirable features and characteristics of the system and method for detecting potential vehicle operator incapacitation based on operator physiological state data will become apparent from the subsequent detailed description and the appended claims, taken in conjunction with the accompanying drawings and the preceding background.

### BRIEF DESCRIPTION OF THE DRAWINGS

The present invention will hereinafter be described in conjunction with the following drawing figures, wherein like numerals denote like elements, and wherein:

FIG. 1 is a block diagram representation of a system for implementing detection of potential vehicle operator incapacitation;

FIG. 2 is a block diagram representation of a vehicle including an embodiment of a vehicle operator incapacitation detection system;

FIG. 3 is a flowchart representation of an exemplary embodiment of a method of detecting potential vehicle operator incapacitation based on operator actigraphy state data; and

FIG. 4 is a flowchart representation of an exemplary embodiment of a method of detecting potential vehicle operator incapacitation based on operator physiological state data.

### DETAILED DESCRIPTION

The following detailed description is merely exemplary in nature. As used herein, the word “exemplary” means “serving as an example, instance, or illustration.” Thus, any embodiment described herein as “exemplary” is not necessarily to be construed as preferred or advantageous over other embodiments. All of the embodiments described herein are exemplary embodiments provided to enable persons skilled in the art to make or use the invention and not to limit the scope of the invention which is defined by the claims. Furthermore, there is no intention to be bound by any expressed or implied theory presented in the preceding technical field, background, brief summary, or the following detailed description.

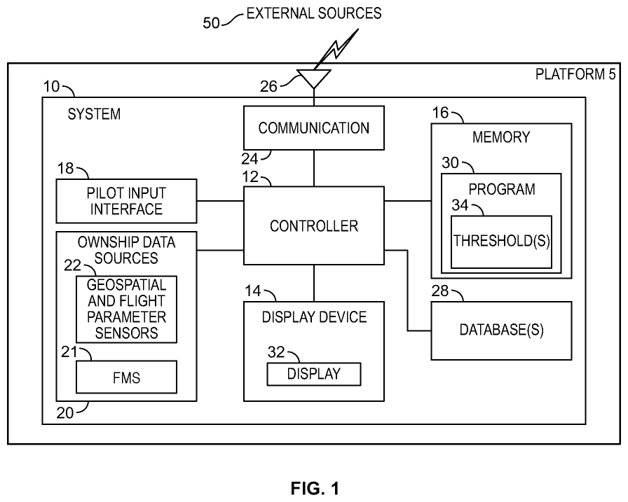

FIG. 1 is a block diagram representation of a system 10 for implementing detection of potential vehicle operator incapacitation based on operator physiological state data (shortened herein to “system” 10), as illustrated in accordance with an exemplary and non-limiting embodiment of the present disclosure. The system 10 may be utilized onboard a mobile platform 5, as described herein. In various embodiments, the mobile platform is an aircraft, which carries or is equipped with the system 10. As schematically depicted in FIG. 1, the system 10 includes the following components or subsystems, each of which may assume the form of a single device or multiple interconnected devices: a controller circuit 12 operationally coupled to: at least one display device 14; computer-readable storage media or memory 16; an optional input interface 18, and ownship data sources 20 including, for example, a flight management system (FMS) 21 and an array of flight system state and geospatial sensors 22.

In various embodiments, the system 10 may be separate from or integrated within: the flight management system (FMS) 21 and/or a flight control system (FCS). Although schematically illustrated in FIG. 1 as a single unit, the individual elements and components of the system 10 can be implemented in a distributed manner utilizing any practical number of physically distinct and operatively interconnected pieces of hardware or equipment. When the system 10 is utilized as described herein, the various components of the system 10 will typically all be located onboard the mobile platform 5.

The term “controller circuit” (and its simplification, “controller”), broadly encompasses those components utilized to carry-out or otherwise support the processing functionalities of the system 10. Accordingly, the controller circuit 12 can encompass or may be associated with a programmable logic array, application specific integrated circuit or other similar firmware, as well as any number of individual processors, flight control computers, navigational equipment pieces, computer-readable memories (including or in addition to the memory 16), power supplies, storage devices, interface cards, and other standardized components. In various embodiments, the controller circuit 12 embodies one or more processors operationally coupled to data storage having stored therein at least one firmware or software program (generally, computer-readable instructions that embody an algorithm) for carrying-out the various process tasks, calculations, and control/display functions described herein. During operation, the controller circuit 12 may be programmed with and execute the at least one firmware or software program, for example, a program 30, that embodies an algorithm described herein for implementing detection of potential vehicle operator incapacitation based on operator physiological state data on a mobile platform 5, where the mobile platform 5 is an aircraft, and to accordingly perform the various process steps, tasks, calculations, and control/display functions described herein.

The controller circuit 12 may exchange data, including real-time wireless data, with one or more external sources 50 to support operation of the system 10 in embodiments. In this case, bidirectional wireless data exchange may occur over a communications network, such as a public or private network implemented in accordance with Transmission Control Protocol/Internet Protocol architectures or other conventional protocol standards. Encryption and mutual authentication techniques may be applied, as appropriate, to ensure data security.

The memory 16 is a data storage that can encompass any number and type of storage media suitable for storing computer-readable code or instructions, such as the aforementioned software program 30, as well as other data generally supporting the operation of the system 10. The memory 16 may also store one or more threshold 34 values, for use by an algorithm embodied in software program 30. One or more database(s) 28 are another form of storage media; they may be integrated with memory 16 or separate from it.

In various embodiments, aircraft-specific parameters and information for an aircraft may be stored in the memory 16 or in a database 28 and referenced by the program 30. Non-limiting examples of aircraft-specific information includes an aircraft weight and dimensions, performance capabilities, configuration options, and the like.

Flight parameter sensors and geospatial sensors 22 supply various types of data or measurements to the controller circuit 12 during an aircraft flight. In various embodiments, the geospatial sensors 22 supply, without limitation, one or more of: inertial reference system measurements providing a location, Flight Path Angle (FPA) measurements, airspeed data, groundspeed data (including groundspeed direction), vertical speed data, vertical acceleration data, altitude data, attitude data including pitch data and roll measurements, yaw data, heading information, sensed atmospheric conditions data (including wind speed and direction data), flight path data, flight track data, radar altitude data, and geometric altitude data.

With continued reference to FIG. 1, the display device 14 can include any number and type of image generating devices on which one or more avionic displays 32 may be produced. When the system 10 is utilized for a manned aircraft, the display device 14 may be affixed to the static structure of the Aircraft cockpit as, for example, a Head Down Display (HDD) or Head Up Display (HUD) unit. In various embodiments, the display device 14 may assume the form of a movable display device (e.g., a pilot-worn display device) or a portable display device, such as an Electronic Flight Bag (EFB), a laptop, or a tablet computer carried into the aircraft cockpit by a pilot.

At least one avionic display 32 is generated on the display device 14 during operation of the system 10; the term “avionic display” is synonymous with the term “aircraft-related display” and “cockpit display” and encompasses displays generated in textual, graphical, cartographical, and other formats. The system 10 can generate various types of lateral and vertical avionic displays 32 on which map views and symbology, text annunciations, and other graphics pertaining to flight planning are presented for a pilot to view. The display device 14 is configured to continuously render at least a lateral display showing the aircraft at its current location within the map data. The avionic display 32 generated and controlled by the system 10 can include graphical user interface (GUI) objects and alphanumerical input displays of the type commonly presented on the screens of multifunction control display units (MCDUs), as well as Control Display Units (CDUs) generally. Specifically, embodiments of the avionic displays 32 include one or more two-dimensional (2D) avionic displays, such as a horizontal (i.e., lateral) navigation display or vertical navigation display (i.e., vertical situation display VSD); and/or on one or more three dimensional (3D) avionic displays, such as a Primary Flight Display (PFD) or an exocentric 3D avionic display.

In various embodiments, a human-machine interface is implemented as an integration of a pilot input interface 18 and a display device 14. In various embodiments, the display device 14 is a touch screen display. In various embodiments, the human-machine interface also includes a separate pilot input interface 18 (such as a keyboard, cursor control device, voice input device, or the like), generally operationally coupled to the display device 14. Via various display and graphics systems processes, the controller circuit 12 may command and control a touch screen display device 14 to generate a variety of graphical user interface (GUI) objects or elements described herein, including, for example, buttons, sliders, and the like, which are used to prompt a user to interact with the human-machine interface to provide user input; and for the controller circuit 12 to activate respective functions and provide user feedback, responsive to received user input at the GUI element.

In various embodiments, the system 10 may also include a dedicated communications circuit 24 configured to provide a real-time bidirectional wired and/or wireless data exchange for the controller 12 to communicate with the external sources 50 (including, each of: traffic, air traffic control (ATC), satellite weather sources, ground stations, and the like). In various embodiments, the communications circuit 24 may include a public or private network implemented in accordance with Transmission Control Protocol/Internet Protocol architectures and/or other conventional protocol standards. Encryption and mutual authentication techniques may be applied, as appropriate, to ensure data security. In some embodiments, the communications circuit 24 is integrated within the controller circuit 12, and in other embodiments, the communications circuit 24 is external to the controller circuit 12. When the external source 50 is “traffic,” the communications circuit 24 may incorporate software and/or hardware for communication protocols as needed for traffic collision avoidance (TCAS), automatic dependent surveillance broadcast (ADSB), and enhanced vision systems (EVS).

In certain embodiments of the system 10, the controller circuit 12 and the other components of the system 10 may be integrated within or cooperate with any number and type of systems commonly deployed onboard an aircraft including, for example, an FMS 21.

The disclosed algorithm is embodied in a hardware program or software program (e.g. program 30 in controller circuit 12) and configured to operate when the aircraft is in any phase of flight. The algorithm enables detection of potential vehicle operator incapacitation based on operator physiological state data in an aircraft.

In various embodiments, the provided controller circuit 12, and therefore its program 30 may incorporate the programming instructions for: receiving raw operator physiological state data from a biosensing wearable device of an operator of a vehicle, the raw operator physiological state data including raw operator actigraphy state data; receiving contextual motion data from at least one environmental motion sensor, the contextual motion data being associated with movement of the vehicle; generating a motion filter mask based on the contextual motion data; applying the motion filter mask to the raw operator actigraphy state data to filter the contextual motion data from the raw operator actigraphy state data to generate actual operator actigraphy state data; determining whether the actual operator actigraphy state data is less than an actigraphy threshold; and issuing an operator incapacity alert based on the determination.

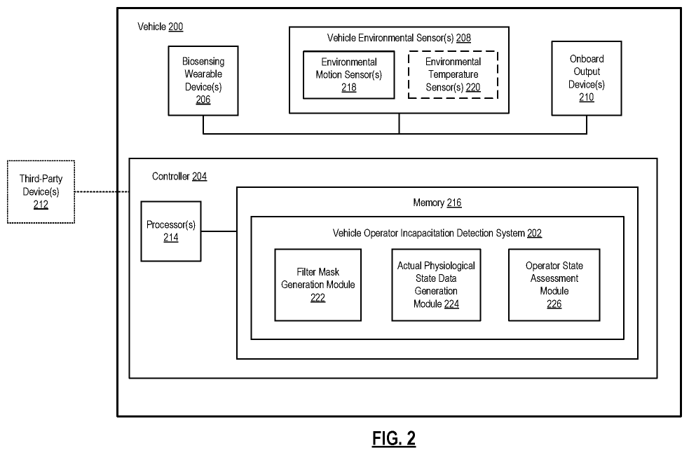

Referring to FIG. 2, a block diagram representation of a vehicle 200 including an embodiment of a vehicle operator incapacitation detection system 202 is shown. Examples of vehicles 200, include, but are not limited to, an aircraft, a watercraft, and a roadway vehicle. In an embodiment, the vehicle 200 is an aircraft having a configuration similar to the platform 5 in FIG. 1.

In an embodiment, the vehicle 200 includes a controller 204, biosensing wearable device(s) 206, vehicle environmental sensor(s) 208, and onboard output device(s) 210. In an embodiment, the controller 204 is configured to be communicatively coupled to third-party device(s) 212. An example of a third-party device 212 is an air traffic controller (ATC) device. The controller 204 includes processor(s) 214 and a memory 216. The memory 216 includes an embodiment of the vehicle operator incapacitation detection system 202. The vehicle 200 may include additional components that facilitate operation of the vehicle 200.

There may be one or more operators of the vehicle 200. Each of the one or more operators of the vehicle 200 wears a biosensing wearable device 206. Examples of biosensing wearable devices 206 include, but are not limited to, a biosensing wristband and a biosensing garment. Each biosensing wearable device 206 includes one or more sensors. Each of the one or more sensors are configured to sense raw operator physiological state data of the operator wearing the wearable device 206. Examples of sensors include, but are not limited to, a motion sensor, a heart rate detector, and a skin temperature sensor. In an embodiment, the raw operator physiological state data includes raw operator actigraphy state data. In an embodiment, the raw operator physiological state data includes raw operator actigraphy state data and one or more of heart rate state data and raw operator electrodermal state data.

The vehicle operator incapacitation detection system 202 is configured to receive the raw operator physiological state data from the biosensing wearable device 206 of the operator. In an embodiment, the vehicle operator incapacitation detection system 202 is configured to receive raw operator actigraphy state data from the biosensing wearable device 206. In an embodiment, the vehicle operator incapacitation detection system 202 is configured to receive raw operator actigraphy state data and one or more of heart rate state data and raw operator electrodermal state data from the biosensing wearable device 206.

The vehicle environmental sensors 208 are configured to sense vehicle environment data associated with the vehicle 200. The vehicle environment sensors 208 include environmental motion sensors 218. In an embodiment, the environmental motion sensors 218 are a component of a vehicle inertial sensor. In an embodiment, the environmental motion sensors 218 are a component of a portable device. In an embodiment, the environmental motion sensors 218 are a component of a phone. In an embodiment, the vehicle environment sensors 208 include environmental motion sensors 218 and environmental temperature sensors 220.

The vehicle operator incapacitation detection system 202 is configured to receive contextual motion data from the environmental motion sensors 218. The contextual motion data represents the motion of the vehicle 200. In an embodiment, the vehicle operator incapacitation detection system 202 is configured to receive contextual motion data from the environmental motion sensors 218 and contextual temperature data from the environmental temperature sensors 220. The contextual temperature data represents a temperature of the interior of the vehicle 200.

The vehicle 200 includes one or more onboard output devices 210. The onboard output device 210 is configured to issue an operator incapacity alert. If the vehicle operator incapacitation detection system 202 determines that that there is a potential vehicle operator incapacity situation, the vehicle operator incapacitation detection system 202 issues a command to the onboard output device 210 to issue the operator incapacity alert. In an embodiment, the onboard output device 210 is a vehicle display device and the operator incapacity alert is a visual alert on the vehicle display device. In an embodiment, the onboard output device 210 is a vehicle acoustic device and the operator incapacity alert is an acoustic alert via the vehicle acoustic device. In an embodiment, the onboard output device 210 is a vehicle haptic device and the operator incapacity alert is a haptic alert via the vehicle haptic device. In an embodiment, if the vehicle operator incapacitation detection system 202 determines that that there is a potential vehicle operator incapacity situation, the vehicle operator incapacitation detection system 202 issues a command to a third-party device 212 to issue the operator incapacity alert. An example of the third-party device 212 is an ATC display device.

In an embodiment, the vehicle operator incapacitation detection system 202 includes a filter mask generation module 222, an actual physiological state data determination module 224, and an operator state assessment module 226. The vehicle operator incapacitation detection system 202 may include additional components that facilitate operation of the vehicle operator incapacitation detection system 202.

The filter mask generation module 222 is configured to receive contextual motion data from the environmental motion sensors 218. The contextual motion data represents movement of the vehicle 200. The filter mask generation module 222 is configured to generate a motion filter mask based on the received contextual motion data. In an embodiment, the filter mask generation module 222 is configured to receive contextual temperature data from the environmental temperature sensor 220. The contextual temperature data represents the interior temperature of the vehicle 200. The filter mask generation module 222 is configured to generate a temperature filter mask based on the received contextual temperature data.

The actual physiological state generation data generation module 224 is configured to receive raw operator physiological state data from the biosensing wearable device 206 worn by an operator of the vehicle 200. The raw operator physiological state data includes raw operator actigraphy state data. The raw operator actigraphy state data received from the biosensing wearable device 206 may be impacted by the movement of the vehicle 200. As a result, the raw operator actigraphy state data may not accurately reflect movements or the intensity of the movements of the operator of the vehicle 200. For example, in cases where the vehicle 200 is a roadway vehicle, a large bump on a road may result in vehicle motion that may impact the accuracy of the operator actigraphy state data sensed by the motion sensor of the biosensing wearable device 206. In another example, in cases where the vehicle 200 is an aircraft, air turbulence encountered during an aircraft flight may result in vehicle motion that may impact the accuracy of the operator actigraphy state sensed by the motion sensor of the biosensing wearable device 206. Motion that is an artifact of an environmental state of vehicle motion may distort the accuracy of the operator actigraphy state data sensed by the biosensing wearable device 206 of the operator of a vehicle 200.

The actual physiological state generation data generation module 224 is configured to receive the motion filter mask from the filter mask generation module 222. The actual physiological state generation data generation module 224 is configured to apply the motion filter mask to the raw operator actigraphy state data to filter the contextual motion data associated with the movement of the vehicle 200 from the raw operator actigraphy state data generated by the biosensing wearable device 206 to generate the actual operator actigraphy state data. The application of the motion filter mask to the raw operator actigraphy state data operates to remove artifacts of an environmental state of vehicle motion from the raw operator actigraphy state data to generate the actual operator actigraphy state data.

In an embodiment, the actual physiological state generation data generation module 224 is configured to receive raw operator electrodermal state data from the biosensing wearable device 206 worn by the operator of the vehicle 200. The raw operator electrodermal state data generated by the biosensing wearable device 206 may be impacted by an interior temperature of the vehicle 200. As a result, the raw operator electrodermal state data may not accurately reflect the body temperature of the operator of the vehicle 200.

The actual physiological state generation data generation module 224 is configured to receive the temperature filter mask from the filter mask generation module 222. The actual physiological state generation data generation module 224 is configured to apply the temperature filter mask to the raw operator temperature state data to filter the contextual temperature data associated with the interior temperature of the vehicle 200 from the raw operator temperature state data to generate the actual operator temperature state data. The application of the temperature filter mask to the raw operator temperature state data operates to remove artifacts of an environmental state of vehicle interior temperature from the raw operator temperature state data generated by the biosensing wearable device 206 to generate the actual operator temperature state data.

In an embodiment, the actual physiological state generation data generation module 224 is configured to receive operator heart rate state data from the biosensing wearable device 206 worn by the operator of the vehicle 200.

The operator state assessment module 226 is configured to receive the actual operator actigraphy state data from the actual physiological state generation module 220. The operator state assessment module 226 is configured to determine whether the actual operator actigraphy state data is less than an actigraphy threshold. When the actual operator actigraphy state data is less than the actigraphy threshold, it is an indication that the intensity of the movement of the operator of the vehicle 200 is relatively low and indicative of a potential operator incapacity situation. When the actual operator actigraphy state data is greater than the actigraphy threshold, it is an indication that the intensity of the movement of the operator of the vehicle 200 sufficiently high that the operator performance appears to be normal.

If the operator state assessment module 226 determines that the actual operator actigraphy state data is less than the actigraphy threshold, the operator state assessment module 226 determines that the operator of the vehicle 200 may be incapacitated and issues an operator incapacity alert. The operator incapacity alert is issued via an onboard output device 210. In an embodiment, the onboard output device 210 is a vehicle display device and the operator incapacity alert is issued as a visual alert on the vehicle display device. In an embodiment, the onboard output device 210 is a vehicle acoustic device and the operator incapacity alert is issued as an acoustic alert via the vehicle acoustic device. In an embodiment, the onboard output device 210 is a vehicle haptic device and the operator incapacity alert is issued as a haptic alert via the vehicle haptic device. In an embodiment, the operator incapacity alert is issued as one or more of a visual alert, an acoustic alert, and a haptic alert.

In an embodiment, the operator state assessment module 226 is configured to issue the operator incapacity alert to a third-party device 212. An example of a third-party device is a ATC display device. In an embodiment, operator state assessment module 226 is configured to implement a vehicle safety action in response to a potential operator incapacity situation. In an embodiment, operator state assessment module 226 is configured to implement a vehicle safety action in response to a determination that the actual operator actigraphy state is less than the actigraphy threshold. Examples of vehicle safety action responses include, but are not limited to, placing the vehicle 200 in a semi-automatic mode and in an automatic mode.

In an embodiment, operator state assessment module 226 is configured to receive the actual operator electrodermal state data from the actual physiological state generation module 220. The operator state assessment module 226 is configured to determine whether the actual operator electrodermal state data is less than a temperature threshold. When the operator electrodermal state data actual is less than the temperature threshold, relatively low temperature may be indicative of a potential operator incapacity situation. When the operator electrodermal state data actual is greater than the temperature threshold, it is an indication that the body temperature may not be a factor in determining whether there is a potential vehicle operator incapacity situation.

In an embodiment, operator state assessment module 226 is configured to receive the operator heart rate state data from the actual physiological state generation module 224. The operator state assessment module 226 is configured to determine whether the operator heart rate state data is less than a heart rate threshold. When the operator heart rate state data is less than the heart rate threshold, relatively low heart rate may be indicative of a potential operator incapacity situation. When the operator heart rate state data is greater than the heart rate threshold, it is an indication that the heart rate may not be a factor in determining whether there is a potential vehicle operator incapacity situation.

In an embodiment, the operator state assessment module 226 is configured to receive the actual operator actigraphy state data and the actual operator electrodermal state data from the actual physiological state generation module 220 and apply a first weight to a determination regarding whether the actual operator actigraphy state data is less than the actigraphy threshold and a second weight to a determination regarding whether the actual operator electrodermal state data is less than the temperature threshold, where the first weight is greater than the second weight. The operator state assessment module 226 is configured to make an assessment regarding a potential vehicle operator incapacity situation and issue an operator incapacity alert based on the assessment of the weighted determinations. In an embodiment, operator state assessment module 226 is configured to implement a vehicle safety action based on the assessment of the weighted determinations.

In an embodiment, the operator state assessment module 226 is configured to receive the actual operator actigraphy state data, the actual operator electrodermal state data, and the operator heart rate data from the actual physiological state generation module 224 and apply a first weight to a determination regarding whether the actual operator actigraphy state data is less than the actigraphy threshold, a second weight to a determination regarding whether the actual operator electrodermal state data is less than the temperature threshold, and a third weight to a determination regarding whether the operator heart rate state data is less than the heart rate threshold. The first weight is greater than the second weight and the third weight. In an embodiment, the second weight is greater than the third weight. In an embodiment, the third weight is greater than the second weight. The operator state assessment module 226 is configured to make an assessment regarding a potential vehicle operation incapacity situation and issue an operator incapacity alert based on the assessment of the weighted determinations. In an embodiment, operator state assessment module 226 is configured to implement a vehicle safety action based on the assessment of the weighted determinations.

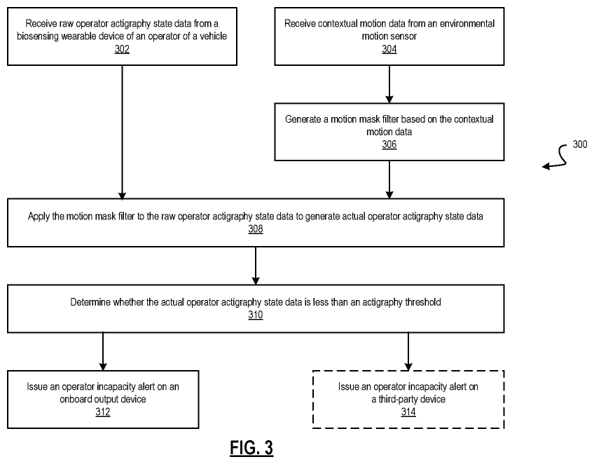

Referring to FIG. 3, a flowchart representation of an exemplary embodiment of a method 300 of detecting potential vehicle operator incapacitation based on operator actigraphy state data is shown. The method 300 is implemented by an embodiment of the of a vehicle operator incapacitation detection system 202.

At 302, the vehicle operator incapacitation detection system 202 receives raw operator actigraphy state data from a biosensing wearable device 206 of an operator of a vehicle 200. The raw operator actigraphy state data is contaminated by environmental dynamics, such as for example, the motion of the vehicle 200. Examples of biosensing wearable devices 206 include, but are not limited to, a biosensing wristband and a biosensing garment. Examples of the vehicle include, but are not limited to an aircraft, a watercraft, and a roadway vehicle.

At 304, the vehicle operator incapacitation detection system 202 receives contextual motion data from an environmental motion sensor 220. In an embodiment, the vehicle operator incapacitation detection system 202 receives the contextual motion data from a vehicle inertial sensor including the environment motion sensor 220. In an embodiment, the vehicle operator incapacitation detection system 202 receives the contextual motion data from a portable device including the environment motion sensor 220. In an embodiment, the vehicle operator incapacitation detection system 202 receives the contextual motion data from a phone including the environment motion sensor 220. An example of a motion sensor is an accelerometer. The vehicle operator incapacitation detection system 202 receives the raw operator actigraphy state data from the biosensing wearable device 206 of the operator of the vehicle 200 and the contextual motion data from an environmental motion sensor 220 at approximately the same time.

At 306, the vehicle operator incapacitation detection system 202 generates a motion mask filter based on the contextual motion data. At 308, the vehicle operator incapacitation detection system 202 applies the motion mask filter to the raw operator actigraphy state data to generate actual operator actigraphy state data. The application of the motion mask filter operates to suppress environmental dynamics in raw operator actigraphy state data so that the actual operator actigraphy state data is uncontaminated by the environmental dynamics.

At 310, the vehicle operator incapacitation detection system 202 determines whether the actual operator actigraphy state data is less than an actigraphy threshold. When the actual operator actigraphy state data is less than the actigraphy threshold, it is an indication that the intensity of the movement of the operator is relatively low and indicative of a potential operator incapacity situation. When the actual operator actigraphy state data is greater than the actigraphy threshold, it is an indication that the intensity of the movement of the operator is sufficiently high that the operator performance appears to be normal.

At 312, the vehicle operator incapacitation detection system 202 issues an operator incapacity alert via an onboard output device 210 based on the determination made at 310 that there is a potential operator incapacity situation. In an embodiment, the onboard output device 210 is a vehicle display device and the operator incapacity alert is used as a visual alert on the vehicle display device. In an embodiment, the onboard output device 210 is a vehicle acoustic device and the operator incapacity alert is used as an acoustic alert via the vehicle acoustic device. In an embodiment, the onboard output device 210 is a vehicle haptic device and the operator incapacity alert is issued as a haptic alert via the vehicle haptic device. In an embodiment, the operator incapacity alert is issued as one or more of a visual alert, an acoustic alert, and a haptic alert.

In an embodiment, at 314, the vehicle operator incapacitation detection system 202 issues an operator incapacity alert to a third-party device 212 based on the determination made at 310 that there is a potential operator incapacity situation. An example of a third-party device is a ATC display device.

In an embodiment vehicle operator incapacitation detection system 202 is configured to implement a vehicle safety action based on the determination made at 310 that there is a potential operator incapacity situation. Examples of vehicle safety actions include, but are not limited to, placing the vehicle 200 in a semi-automatic mode and placing the vehicle 200 an automatic mode.

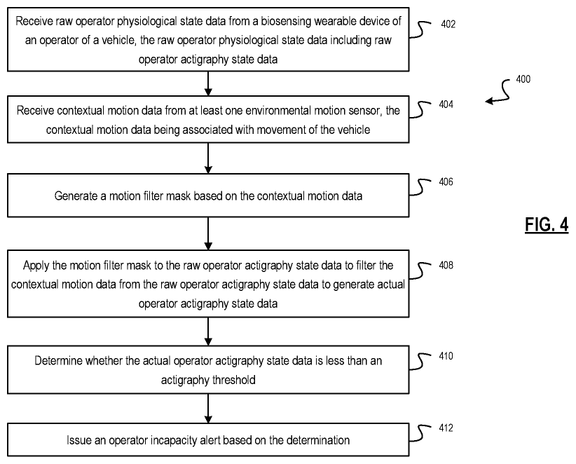

Referring to FIG. 4, a flowchart representation of an exemplary embodiment of a method 400 of detecting potential vehicle operator incapacitation based on operator physiological state data is shown. The method 400 is implemented by an embodiment of a vehicle operator incapacitation detection system 202.

At 402, raw operator physiological state data is received from a biosensing wearable device 206 of an operator of a vehicle 200. The raw operator physiological state data includes raw operator actigraphy state data. At 404, contextual motion data is received from at least one environmental motion sensor 218. The contextual motion data is associated with movement of the vehicle 200. At 406, a motion filter mask is generated based on the contextual motion data. At 408, the motion filter mask is applied to the raw operator actigraphy state data to filter the contextual motion data from the raw operator actigraphy state data to generate actual operator actigraphy state data. At 410, a determination is made regarding whether the actual operator actigraphy state data is less than an actigraphy threshold. At 412, an operator incapacity alert is issued based on the determination.

The physiological states of an operator of a vehicle 200 provides insight into whether the operator of the vehicle 200 may be potentially incapacitated. The operator of the vehicle 200 often wears biosensing wearable devices 206 that are configured to provide operator physiological state data that can be used to estimate the physiological state of an operator (for example, potential operator incapacity). An example of operator physiological state data is actigraphy. Actigraphy is a measurable variable associated with a body part of the vehicle operator that can be obtained from an accelerometer of the biosensing wearable device. Actigraphy is an indicator of the intensity of movement of the body part being monitored by the wearable device and is considered to be a reliable indicator of potential vehicle operator incapacitation.

A technical challenge associated with the use of raw operator actigraphy state data is that environmental dynamics associated with the movement of the vehicle 200 may negatively impact the reliability of the operator actigraphy state data received from the biosensing wearable device 206 of the operator of the vehicle 200. For example, a large bump on a road or turbulence encountered during an aircraft flight may be misinterpreted as vehicle operator motion, when the source of that motion is just an artifact of an environmental state of vehicle motion. Environment dynamics may impact the accuracy of the operator actigraphy state data generated by the biosensing wearable device 206 and lead to unreliable detection of potential vehicle operator incapacitation.

A technical solution is to detect contextual motion data, where the contextual motion data is associated with movement of the vehicle 200, generate a motion filter mask based on the contextual motion data, and apply the motion filter mask to the raw operator actigraphy state data generated by the biosensing wearable device 206 of the operator to filter the contextual motion data from the raw operator actigraphy data to generate actual operator actigraphy state data. The actual operator actigraphy state data is relatively more accurate than the raw operator actigraphy state data and a more reliable indicator of potential vehicle operator incapacitation.

Those of skill in the art will appreciate that the various illustrative logical blocks, modules, circuits, and algorithm steps described in connection with the embodiments disclosed herein may be implemented as electronic hardware, computer software, or combinations of both. Some of the embodiments and implementations are described above in terms of functional and/or logical block components (or modules) and various processing steps. However, it should be appreciated that such block components (or modules) may be realized by any number of hardware, software, and/or firmware components configured to perform the specified functions. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present invention. For example, an embodiment of a system or a component may employ various integrated circuit components, e.g., memory elements, digital signal processing elements, logic elements, look-up tables, or the like, which may carry out a variety of functions under the control of one or more microprocessors or other control devices. In addition, those skilled in the art will appreciate that embodiments described herein are merely exemplary implementations.

The various illustrative logical blocks, modules, and circuits described in connection with the embodiments disclosed herein may be implemented or performed with a general-purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

The steps of a method or algorithm described in connection with the embodiments disclosed herein may be embodied directly in hardware, in a software module executed by a processor, or in a combination of the two. A software module may reside in RAM memory, flash memory, ROM memory, EPROM memory, EEPROM memory, registers, hard disk, a removable disk, a CD-ROM, or any other form of storage medium known in the art. An exemplary storage medium is coupled to the processor such that the processor can read information from, and write information to, the storage medium. In the alternative, the storage medium may be integral to the processor. The processor and the storage medium may reside in an ASIC.

Techniques and technologies may be described herein in terms of functional and/or logical block components, and with reference to symbolic representations of operations, processing tasks, and functions that may be performed by various computing components or devices. Such operations, tasks, and functions are sometimes referred to as being computer-executed, computerized, software-implemented, or computer-implemented. In practice, one or more processor devices can carry out the described operations, tasks, and functions by manipulating electrical signals representing data bits at memory locations in the system memory, as well as other processing of signals. The memory locations where data bits are maintained are physical locations that have particular electrical, magnetic, optical, or organic properties corresponding to the data bits. It should be appreciated that the various block components shown in the figures may be realized by any number of hardware, software, and/or firmware components configured to perform the specified functions. For example, an embodiment of a system or a component may employ various integrated circuit components, e.g., memory elements, digital signal processing elements, logic elements, look-up tables, or the like, which may carry out a variety of functions under the control of one or more microprocessors or other control devices.

When implemented in software or firmware, various elements of the systems described herein are essentially the code segments or instructions that perform the various tasks. The program or code segments can be stored in a processor-readable medium or transmitted by a computer data signal embodied in a carrier wave over a transmission medium or communication path. The “computer-readable medium”, “processor-readable medium”, or “machine-readable medium” may include any medium that can store or transfer information. Examples of the processor-readable medium include an electronic circuit, a semiconductor memory device, a ROM, a flash memory, an erasable ROM (EROM), a floppy diskette, a CD-ROM, an optical disk, a hard disk, a fiber optic medium, a radio frequency (RF) link, or the like. The computer data signal may include any signal that can propagate over a transmission medium such as electronic network channels, optical fibers, air, electromagnetic paths, or RF links. The code segments may be downloaded via computer networks such as the Internet, an intranet, a LAN, or the like.

Some of the functional units described in this specification have been referred to as “modules” in order to more particularly emphasize their implementation independence. For example, functionality referred to herein as a module may be implemented wholly, or partially, as a hardware circuit comprising custom VLSI circuits or gate arrays, off-the-shelf semiconductors such as logic chips, transistors, or other discrete components. A module may also be implemented in programmable hardware devices such as field programmable gate arrays, programmable array logic, programmable logic devices, or the like. Modules may also be implemented in software for execution by various types of processors. An identified module of executable code may, for instance, comprise one or more physical or logical modules of computer instructions that may, for instance, be organized as an object, procedure, or function. Nevertheless, the executables of an identified module need not be physically located together but may comprise disparate instructions stored in different locations that, when joined logically together, comprise the module and achieve the stated purpose for the module. Indeed, a module of executable code may be a single instruction, or many instructions, and may even be distributed over several different code segments, among different programs, and across several memory devices. Similarly, operational data may be embodied in any suitable form and organized within any suitable type of data structure. The operational data may be collected as a single data set or may be distributed over different locations including over different storage devices, and may exist, at least partially, merely as electronic signals on a system or network.

In this document, relational terms such as first and second, and the like may be used solely to distinguish one entity or action from another entity or action without necessarily requiring or implying any actual such relationship or order between such entities or actions. Numerical ordinals such as “first,” “second,” “third,” etc. simply denote different singles of a plurality and do not imply any order or sequence unless specifically defined by the claim language. The sequence of the text in any of the claims does not imply that process steps must be performed in a temporal or logical order according to such sequence unless it is specifically defined by the language of the claim. The process steps may be interchanged in any order without departing from the scope of the invention as long as such an interchange does not contradict the claim language and is not logically nonsensical.

Furthermore, depending on the context, words such as “connect” or “coupled to” used in describing a relationship between different elements do not imply that a direct physical connection must be made between these elements. For example, two elements may be connected to each other physically, electronically, logically, or in any other manner, through one or more additional elements.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention. It being understood that various changes may be made in the function and arrangement of elements described in an exemplary embodiment without departing from the scope of the invention as set forth in the appended claims.

What is claimed is:

1. A system comprising:
a processor; and
a memory, the memory comprising instructions that upon execution by the processor, cause the processor to:
receive raw operator physiological state data from a biosensing wearable device of an operator of a vehicle, the raw operator physiological state data comprising raw operator actigraphy state data;
receive contextual motion data from at least one environmental motion sensor, the contextual motion data being associated with movement of the vehicle;
generate a motion filter mask based on the contextual motion data;
apply the motion filter mask to the raw operator actigraphy state data to filter the contextual motion data from the raw operator actigraphy state data to generate actual operator actigraphy state data;
determine whether the actual operator actigraphy state data is less than an actigraphy threshold; and
issue an operator incapacity alert based on the determination.

2. The system of claim 1, wherein the memory comprises further instructions that upon execution by the processor, cause the processor to receive the raw operator actigraphy state data from a motion sensor of the biosensing wearable device.

3. The system of claim 1, wherein the memory comprises further instructions that upon execution by the processor, cause the processor to receive the raw operator physiological state data from the biosensing wearable device, wherein the biosensing wearable device is one of a biosensing wristband and a biosensing garment.

4. The system of claim 1, wherein the memory comprises further instructions that upon execution by the processor, cause the processor to receive the contextual motion data from one or more of a vehicle inertial sensor including the at least one environmental motion sensor, a portable device including the at least one environmental motion sensor, and a phone including the at least one environmental motion sensor.

5. The system of claim 1, wherein the memory comprises further instructions that upon execution by the processor, cause the processor to:
receive the raw operator physiological state data from the biosensing wearable device of the operator of the vehicle, the raw operator physiological state data comprising operator heart rate state data;
determine whether the operator heart rate state data is less than a heart rate threshold; and
issue the operator incapacity alert based on the determination.

6. The system of claim 1, wherein the memory comprises further instructions that upon execution by the processor, cause the processor to:
receive the raw operator physiological state data from the biosensing wearable device of the operator of the vehicle, the raw operator physiological state data comprising raw operator electrodermal state data;
receive contextual temperature data from at least one environmental temperature sensor, the contextual temperature data being associated with an interior temperature of the vehicle;
generate a temperature filter mask based on the contextual temperature data;
apply the temperature filter mask to the raw operator electrodermal state data to filter the contextual temperature data from the raw operator electrodermal state data to generate actual operator electrodermal state data;
determine whether the actual operator electrodermal state data is less than a temperature threshold; and
issue the operator incapacity alert based on the determination.

7. The system of claim 1, wherein the memory comprises further instructions that upon execution by the processor, cause the processor to receive the raw operator physiological state data from the biosensing wearable device of the operator of the vehicle, wherein the vehicle is one of an aircraft, a watercraft, and a roadway vehicle.

8. The system of claim 1, wherein the memory comprises further instructions that upon execution by the processor, cause the processor to issue the operator incapacity alert, wherein the operator incapacity alert is at least one of a visual alert on a vehicle display device, an acoustic alert, and a haptic alert.

9. The system of claim 1, wherein the memory comprises further instructions that upon execution by the processor, cause the processor to implement a vehicle safety action responsive to a determination that the actual operator actigraphy state data is less than the actigraphy threshold.

10. The system of claim 1, wherein the memory comprises further instructions that upon execution by the processor, cause the processor to issue the operator incapacity alert to a third-party device.

11. A method comprising:
receiving raw operator physiological state data from a biosensing wearable device of an operator of a vehicle, the raw operator physiological state data comprising raw operator actigraphy state data;
receiving contextual motion data from at least one environmental motion sensor, the contextual motion data being associated with movement of the vehicle;
generating a motion filter mask based on the contextual motion data;
applying the motion filter mask to the raw operator actigraphy state data to filter the contextual motion data from the raw operator actigraphy state data to generate actual operator actigraphy state data;
determining whether the actual operator actigraphy state data is less than an actigraphy threshold; and
issuing an operator incapacity alert based on the determination.

12. The method of claim 11, further comprising receiving the raw operator actigraphy state data from a motion sensor of the biosensing wearable device.

13. The method of claim 11, further comprising receiving the raw operator physiological state data from the biosensing wearable device, wherein the biosensing wearable device is one of a biosensing wristband and a biosensing garment.

14. The method of claim 11, further comprising receiving the contextual motion data from one or more of a vehicle inertial sensor including the at least one environmental motion sensor, a portable device including the at least one environmental motion sensor, and a phone including the at least one environmental motion sensor.

15. The method of claim 11, further comprising:
receiving the raw operator physiological state data from the biosensing wearable device of the operator of the vehicle, the raw operator physiological state data comprising operator heart rate state data;
determining whether the operator heart rate state data is less than a heart rate threshold; and
issuing the operator incapacity alert based on the determination.

16. The method of claim 11, further comprising:
receiving the raw operator physiological state data from the biosensing wearable device of the operator of the vehicle, the raw operator physiological state data comprising raw operator electrodermal state data;
receiving contextual temperature data from at least one environmental temperature sensor, the contextual temperature data being associated with an interior temperature of the vehicle;
generating a temperature filter mask based on the contextual temperature data;
applying the temperature filter mask to the raw operator electrodermal state data to filter the contextual temperature data from the raw operator electrodermal state data to generate actual operator electrodermal state data;
determining whether the actual operator electrodermal state data is less than a temperature threshold; and
issuing the operator incapacity alert based on the determination.

17. The method of claim 11, further comprising receiving the raw operator physiological state data from the biosensing wearable device of the operator of the vehicle, wherein the vehicle is one of an aircraft, a watercraft, and a roadway vehicle.

18. The method of claim 11, further comprising issuing the operator incapacity alert, wherein the operator incapacity alert is at least one of a visual alert on a vehicle display device, an acoustic alert, and a haptic alert.

19. The method of claim 11, further comprising implementing a vehicle safety action responsive to a determination that the actual operator actigraphy state data is less than the actigraphy threshold.

20. The method of claim 11, further comprising issuing the operator incapacity alert to a third-party device.
